(19) United States
(12) Patent Application Publication
(10) Pub. No.: US 2025/0281486 A1
(43) Pub. Date: Sep. 11, 2025

(54) THERAPEUTIC STRATEGIES TO MANAGE FACIAL CONTRACTURES POST INJURY

(71) Applicant: THE TRUSTEES OF INDIANA UNIVERSITY, Bloomington, IN (US)

(72) Inventors: CHANDAN K. SEN, Indianapolis, IN (US); SASHWATI ROY, Indianapolis, IN (US); MOHAMED SALAH ABD EL MAKSOOD EL MASRY, Carmel, IN (US)

(21) Appl. No.: 18/861,236

(22) PCT Filed: Apr. 25, 2023
(86) PCT No.: PCT/US2023/066164

§ 371 Date: Oct. 29, 2024

Related U.S. Application Data
(60) Provisional application No. 63/338,590, filed on May 5, 2022.

(51) Int. Cl. A61K 31/506; A61K 9/00; A61K 31/195; A61L 26/00; A61P 17/02

(52) U.S. Cl. CPC A61K 31/506; A61K 9/0021; A61K 31/195; A61L 26/0033; A61P 17/02; A61L 2300/252; A61L 2430/34

(57) ABSTRACT

Compositions and methods are provided for reducing facial contracture and to alleviate deficits associated with thermal injury of the face, including ectropion (epithelial-ocular junction), eversion of the lip (epithelial-oral junction), and oral incompetence (drooling). In accordance with one embodiment such compositions are used in conjunction with known treatments for use on burn patients.

### CROSS-REFERENCE TO RELATED APPLICATIONS

This application claims priority to U.S. Provisional Patent Application No. 63/338,590 filed on May 5, 2022, the disclosure of which is expressly incorporated herein.

### BACKGROUND OF THE DISCLOSURE

The face is often the seat of recognition for a human being, It is perhaps the most powerful “channel” of nonverbal communication and living with a distortion in the appearance of one's face as a result of injury due to burns or other trauma is challenging. In the United States, about 1.25 million people are treated each year for burns, and 40,000 are hospitalized for the treatment of these injuries resulting in high medical costs of approximately $7.9 billion per year. One source of maxillofacial thermal injuries arises from exposure to the heat of an explosive blast that causes flash burn of an unprotected face. These burns involve the skin, underlying muscle and often even the bone. Because of the high heat involved in explosive blasts, sixth degree burns are common. Maxillofacial thermal or other injuries cause major contractures viewed as severe scarring that burdens the subject socially, emotionally, psychologically and functionally. Critical and unique functional deficits in thermal injury of the face induced by such skin contracture include ectropion (epithelial-ocular junction), eversion of the lip (epithelial-oral junction) and oral incompetence (drooling).

Facial skin, unlike skin of the rest of the body, is of neural crest origin (as opposed to mesodermal origin in other parts of the body). Given this difference, the development of facial scar was investigated and found by applicant to follow a mechanistically unique path where contractile elements of the skeletal (mostly) and cardiac (few) muscles are expressed in the skin. Compared to scar response in other parts of the body, facial scar contractures are much more severe. Existing scar therapies originate from studies on skin of mesodermal origin and are not beneficial to applications in the context of facial contractures. Given the lack of appropriate face burn models to test therapies, this difference could not be previously distinguished. Accordingly the present disclosure is directed to novel methods for treating injury to facial skin resulting from burns or other trauma. The methods could potentially be extended to manage contractures in other parts of the body.

### SUMMARY

In accordance with one embodiment of the present disclosure, a method of treating patients suffering from the effects of maxillofacial injuries is provided, including for example, maxillofacial thermal injuries that result in severe facial contractures that cause disfiguration. Applicant has discovered that facial contractures follow a different pathway than scar development in other skin tissue on the body. This alternate pathways leads to facial scar contractures being much more severe compared to scar response in other parts of the body. In damaged facial skin, contractile elements of the skeletal (mostly) and cardiac (few) muscles are expressed in the cells involved in repairing the damage tissue, leading to severe contractures in the resultant scar tissue. Accordingly, facial scars are more intractable to current strategies for treating scars.

As disclosed herein, applicant's discovery that contractile elements are induced in damaged facial skin led to applicant' proposed novel strategies of treating both existing mature scar (late stage), and for minimizing post injury facial contracture formation (early stage). The methods disclosed herein can reduce contractures associated with existing facial scars and alleviate deficits associated with thermal injury of the face, including reducing ectropion (epithelial-ocular junction), eversion of the lip (epithelial-oral junction) and oral incompetence (drooling).

As disclosed herein drugs that target muscle relaxation are anticipated to help treat facial contractures and decrease the formation of severe contractures during the repair of facial wounds including thermal injuries. In accordance with one embodiment burns to tissue of neural crest origin (e.g., facial skin tissue) are treated by administering one or more anti-myogenic agents in conjunction with one or more muscle relaxants, optionally wherein damages tissue is treated sequentially with an anti-myogenic agent followed by treatment with a muscle relaxant. In accordance with one embodiment such anti-myogenic agents and/or muscle relaxants can be applied topically or injected intradermally into, or adjacent to, the afflicted tissue. In one embodiment multiple injections are administered in one therapeutic treatment, wherein the injection sites are about 1-3 inches apart from one another. Multiple rounds of such therapeutic treatments can be conducted during the course of the therapeutic regimen. In one embodiment after administration of the initial therapeutic treatment of the therapeutic regimen, the treated tissue is covered with a wound dressing comprising hydrolyzed collagen. The wound dressing is subsequently removed prior to each subsequent administration of the therapeutic treatments comprising an anti-myogenic agent and/or muscle relaxant in embodiments where multiple rounds of administration occur. When multiple rounds of therapeutic treatments are administered during the therapeutic regimen, the compositions of the treatments can be the same or can be altered in content. In one embodiment the first administered composition comprises only one or more anti-myogenic agents as the active agent and subsequent administered compositions comprise one or more muscle relaxants with or without the anti-myogenic agents. In one embodiment where the therapeutic regimen includes the use of a wound dressing, any previously applied wound dressing is removed prior to administration of the therapeutic composition (optionally administered via intradermal injection) and a new wound dressing is applied after the administration of the therapeutic composition of the present invention.

In one embodiment the wound dressing is a hydrolyzed collagen based dressing, including for example but not limited to Elastogel™. In one embodiment the hydrolyzed collagen wound dressing comprises the anti-myogenic agents and/or muscle relaxants coated onto the surface of the wound dressing or embedded within the wound dressing. In one embodiment the hydrolyzed collagen based dressing does not contain any anti-myogenic agents and/or muscle relaxants and is simply applied to the surface of the afflicted tissues after administration of a composition comprising the anti-myogenic agents and/or muscle relaxants.

In accordance with one embodiment the anti-myogenic agent and muscle relaxant are administered sequentially, optionally wherein any previously applied wound dressing is removed prior to administration of the composition comprising the anti-myogenic agent and/or muscle relaxant, followed by application of a new hydrolyzed collagen based wound dressing over the treated tissues. In one embodiment the compositions of the present disclosure are administered at an early stage, shortly after the injury (typically within 7 days of the injury, but including up to two weeks to 2 months after the injury). Drugs inhibiting myogenesis have been found to be particularly efficacious in reducing facial contractures during or after the healing of the wound. FDA approved anti-myogenic agents include Nilotinib (Tasigna/AMN107®; Novartis), but other inhibitors of myogenesis known to those skilled in the art and can be used, including, but not limited to, stromal interaction molecule (STIM) inhibitors and estrogen-related receptor alpha inhibitors. Examples of late stage FDA-approved muscle relaxer drugs that can be repurposed include: baclofen, dantrolene, and tizanidine. Additional muscle relaxer drugs suitable for use in the present invention include carisoprodol, chlorzoxazone, cyclobenzaprine, metaxalone, methocarbamol, and orphenadrine.

In accordance with one embodiment a method of treating contractures and mature scar tissue located on the face or neck of mammalian subject (optionally a human subject) is provided, wherein a composition comprising an inhibitor of myogenesis is administered to a subject in need of such treatment in conjunction with the administration of a skeletal muscle relaxant. In one embodiment a method of treating or reducing the formation of facial scar tissue, or treating mature scar tissue, comprises applying to a facial wound or scar tissue a composition comprising an anti-myogenic agent. In one embodiment the composition comprising an anti-myogenic agent is administered via intradermal injection at one or more sites of the afflicted facial tissue, followed by covering the afflicted tissue with a wound dressing that comprises hydrolyzed collagen, including for example Elastogel™. After a predetermined length of time the wound dressing is removed and a composition comprising a muscle relaxant is administered to the afflicted tissue, optionally via intradermal injection, at one or more sites of the afflicted facial tissue, and the afflicted tissue is then covered with a wound dressing that comprises hydrolyzed collagen, including for example Elastogel™. In one embodiment the skeletal muscle relaxant used in accordance with the present disclosure is selected from the group consisting of baclofen, dantrolene, tizanidine. carisoprodol, chlorzoxazone, cyclobenzaprine, metaxalone, methocarbamol, and orphenadrine.

In one embodiment a method is provided for reducing the severity of scar tissue formation on the face or neck of mammalian subject, optionally a human subject, during the healing phase after injury, wherein the method comprises the step of administering a composition comprising an anti-myogenic agent and/or a muscle relaxant to a subject in need of such treatment, optionally wherein said anti-myogenic agent and/or a muscle relaxant are administered via intradermal injection followed by covering the treated tissue with a wound dressing comprising hydrolyzed collagen. In one embodiment the anti-myogenic agent is Nilotinib (Tasigna/AMN107®; Novartis) and the muscle relaxant is baclofen.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCI, and a pH of 7.2-7.4.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but sufficient quantity of the drug to provide the desired effect. Desired effects in the context of the present invention include diminishing the formation of scar tissue, decreasing the amount of collagen present in the scar tissue that forms and/or decreasing scar contracture and/or the surface area of existing scar tissue. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, cats, dogs and other pets) and humans and includes individuals receiving a therapeutic treatment whether or not they are under the direct supervision of a physician.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein a reference to a “face” includes the front part of the head that in humans extends from the forehead to the chin and extends laterally to the sides of the head and includes the ears, mouth, nose, cheeks, and eyes.

As used herein “facial trauma”, also called maxillofacial trauma, is any physical trauma to the face and the neck. Facial trauma can involve soft tissue injuries such as burns, lacerations and bruises, and may include fractures and damage to facial bones such as nasal fractures and fractures of the jaw.

As used herein the term “burn scar contracture” defines a condition where the skin thickens, tightens and loses flexibility after a second or third degree burn.

As defined herein “wound healing” defines a process wherein a living organism replaces destroyed or damaged tissue by newly produced tissue.

As defined herein the term “anti-myogenic agent” encompasses any compound that decreases the ability of tissues or cells to generate skeletal muscular tissue.

As defined herein the term “muscle relaxant” encompasses any compound that decreases skeletal muscle contracture and decreases the muscle tone and includes two major therapeutic groups of relaxants: neuromuscular blockers and spasmolytics.

### Embodiments

Scars form as part of the natural healing process when the dermis is damaged. In response to such damage, the body forms new collagen fibers to mend the damage, resulting in the formation of scar tissue. Scar tissue, if treated appropriately, can typically be remodeled to resemble normal, healthy tissue. However, depending on the extent of the initial injury and healing responses some scars are severe and can be disfiguring.

For example, the heat of an explosive blast causes flash burn of the face, not protected by armor. These burns involve the skin, underlying muscle and often even the bone. Because of the high heat involved, fourth to sixth degree burns are common. Such maxillofacial thermal injuries cause major facial contractures that burdens the subject socially, emotionally, psychologically and functionally. Critical and unique functional deficits in thermal injury of the face include ectropion (epithelial-ocular junction), eversion of the lip (epithelial-oral junction) and oral incompetence (drooling). Compared to scar response in other parts of the body, facial contractures are much more severe.

To develop novel strategies to manage facial contractures, the molecular mechanisms of post-burn facial contractures versus scarring of skin of the back were compared in a pair-matched pre-clinical porcine setting. Facial skin, unlike skin of the rest of the body, is of neural crest origin. Sequencing studies on a severe face burn model led to the discovery that myogenic genes play a unique role in contracture formation in skin tissue of the face. More particularly, applicant has discovered that following a severe burn, skin and related wound cells in the face undergo myogenic conversion resulting in overt contracture that is responsible for the unique characteristics in thermal injury of the face described above. Repair of skin tissue of the face follows a mechanistically unique path where contractile elements of the skeletal (mostly) and cardiac (few) muscles are expressed in the skin. Scarring of the face cannot be readily treated by conventional anti-scarring strategies currently used today. In accordance with the present disclosure the treatment of facial contractures can be managed using two approaches based on the stage of facial wound repair.

One treatment strategy relates to existing mature scar (late stage), where existing scars are treated to alleviate pain and disfigurement associated with the scars. Another method of treatment relates to post injury contracture formation (early stage) and the administration of therapeutics to diminish the formation of scar tissue including decreasing the amount of collagen present in the scar tissue that forms and/or decreasing surface area of the scar tissue.

In accordance with one embodiment methods are provided to enhance the repair of maxillofacial injuries, including thermally damaged facial skin, while minimizing the formation of post injury contractures. In one embodiment the method comprises the administration of an anti-myogenic agent in conjunction with a skeletal muscle relaxant in a therapeutic effective amount. In accordance with one embodiment the agents are administered sequentially, with the anti-myogenic agent being administered first. In one embodiment the anti-myogenic agent is first administered for 1, 2, 3, 6, 8, 12 hours or 1, 2, 3, 4, 5, 6, or 7 days before the skeletal muscle relaxant is administered. The timing and duration of the administration of the first and second agents will be dependent on the extent of the injury and the speed of the healing process. In one embodiment the anti-myogenic agent is administered only after evidence of wound healing is observed (e.g., granulation tissue formation, contractures around edges or other biological markers of healing). In one embodiment the administration of the anti-myogenic agent is discontinued prior to the administration of the skeletal muscle relaxant, with either immediate administration of the skeletal muscle relaxant upon cessation of the anti-myogenic agent or with a 1, 2, 3, 4, 5, 6, 7, 8, 9 or 10 day delay between cessation of the anti-myogenic agent and administration of the skeletal muscle relaxant. In one embodiment the administration of the anti-myogenic agent is reduced, or tapered to an endpoint, upon initiation of administration of the skeletal muscle relaxant. In one embodiment the administration of the anti-myogenic agent is reduced, or tapered to an endpoint, upon initiation of administration of the skeletal muscle relaxant, wherein the muscle relaxant is initially administered at a low dose that is increased as the anti-myogenic agent dose is reduced. In one embodiment the administration of the anti-myogenic agent is continued in the same dosage after initiation of the administration of the skeletal muscle relaxant and both agents are then co-administered throughout the remainder of the treatment process. In one embodiment the anti-myogenic agent is co-administered with the skeletal muscle relaxant at the onset of treatment and throughout treatment. In one embodiment treatment is continued until at least 65%, 75%, 85% or 95% closure of the wound, and in one embodiment treatment is continued until complete closure of the wound.

In accordance with one embodiment a fresh wound dressing is applied to the surface of the wound or scar tissue after each topical application or each intradermal injection of a composition comprising the anti-myogenic agent and/or skeletal muscle relaxant.

In accordance with one embodiment the administration of the anti-myogenic agent and/or skeletal muscle relaxant is conducted in conjunction with the application of a collagen based wound dressing. The collagen wound dressings can be formulated as gels, pads, pastes, or sheets derived from bovine, equine, porcine or avian collagen sources. In one embodiment the wound or scar tissue is first contacted with a collagen based wound dressing prior to the administration of the anti-myogenic agent and/or skeletal muscle relaxant, wherein the wound dressing is removed prior to administration of the anti-myogenic agents and/or muscle relaxants, and a new wound dressing is applied after the administration of the anti-myogenic agents and/or muscle relaxants. Suitable collagen based wound dressings are commercially available and include but are not limited to Elastogel™.

In one embodiment a collagen based wound dressing is used as a vehicle for delivery of the anti-myogenic agent and skeletal muscle relaxant, wherein the anti-myogenic agent and/or skeletal muscle relaxant is coated on, or located within the spaces of the collagen would dressing. In one embodiment, the composition is configured for sustained release of the myogenic agent and/or skeletal muscle relaxant disposed in pores of the collagen matrix. In some embodiments, the composition is configured for sustained release of the anti-myogenic agent and/or skeletal muscle relaxant disposed in the wound dressing over a period of 24, 48, 72 or up to about 96 hours.

In some embodiments, the composition is configured for rapid release of the myogenic agent and/or skeletal muscle relaxant disposed in the wound dressing, optionally wherein release is completed within about 24 hours.

In one embodiment a method of treating mature scars or reducing the formation of facial scars is provided wherein a series of compositions comprising an anti-myogenic agent and/or skeletal muscle relaxant are topically applied or intradermally injected into the surface of the wound/scar followed by covering the treated tissue with a wound dressing. In one embodiment facial burns are treated by the sequential application of a composition comprising an anti-myogenic agent, followed by the application of a composition comprising a skeletal muscle relaxant, wherein the treated tissue is covered with a collagen based wound dressing after each composition is administered. In one embodiment the wound dressing is completely removed before application of the next round of treatment with a composition comprising an anti-myogenic agent and/or skeletal muscle relaxant. In a further embodiment the affected skin tissues are first contacted with a collagen based wound dressing that lacks an anti-myogenic agent or skeletal muscle relaxant, prior to subsequent application of the compositions comprising an anti-myogenic agent and/or skeletal muscle relaxant.

In embodiments where a collagen wound dressing is applied to a wound or scar tissue, the time the wound dressing remains before being removed can vary based on the composition of the applied wound dressing, the release mechanics of the applied wound dressing and whether a series of wound dressings of differing composition will be applied. Accordingly, the wound dressing can be applied for 6, 12, 24, 48, 72 or 96 hours before removal or replacement.

In one embodiment facial burns are treated by the sequential administration of an anti-myogenic agent and a skeletal muscle relaxant, wherein the skeletal muscle relaxant is administered when 75% of the facial burn wound has closed and no signs of infection are present, optionally wherein a new wound dressing is applied after each of the sequential administrations of the anti-myogenic agent and a skeletal muscle relaxant compositions.

In one embodiment the anti-myogenic agent used in accordance with the present invention is selected from the group consisting of Nilotinib (Tasigna/AMN107®; Novartis), stromal interaction molecule (STIM) inhibitors and estrogen-related receptor alpha inhibitors. In one embodiment the muscle relaxant used in accordance with the present invention is selected from the group consisting of baclofen, dantrolene, tizanidine, carisoprodol, chlorzoxazone, cyclobenzaprine, metaxalone, methocarbamol, and orphenadrine. In one embodiment the muscle relaxant used in accordance with the present invention is selected from the group consisting of baclofen, dantrolene, and tizanidine. In one embodiment a method of treating a facial burn comprises administering Nilotinib as the anti-myogenic factor in conjunction with the administration of baclofen as the muscle relaxant.

In one embodiment a pharmaceutical composition is provided for treating maxillofacial injuries, including thermally damaged facial skin, wherein the composition comprises an anti-myogenic agent and a pharmaceutically acceptable carrier. In one embodiment the composition further comprises a skeletal muscle relaxant. In one embodiment the pharmaceutical composition is formulated for administration by any acceptable route including as an injectable or topical formulation. In one embodiment the composition is formulated as a topical cream or ointment that is placed in contact with the external surface of the scar. In one embodiment the topical cream or ointment can be directly applied to the scar tissue, or the scar can be covered in gauze, bandage or other matrix that releases the formulation to the patient's affected skin a time released manner.

In one embodiment the pharmaceutical composition is formulated as an injectable, and the composition is administered by injecting the formulation into the wound edge tissues.

In accordance with one embodiment, drugs that stimulate muscle relaxation will be used to treat mature facial contractures. In one embodiment the subject is a human and the mature facial contracture to be treated is a severe scar resulting from traumatic damage to facial skin. In one embodiment the scar is formed after healing from a burn caused by heat, chemicals, electricity, sunlight, or nuclear radiation. In one embodiment the method of the present disclosure is directed to treating mature contractures formed after thermal damage to human facial skin, including scars formed after healing from second, third, fourth, fifth or sixth degree burns.

One method of treating mature contractures located on the face or neck of mammalian subject comprises the step of administering a composition comprising a skeletal muscle relaxant and a pharmaceutically acceptable carrier to a subject in need of such treatment, optionally in conjunction with administration of an anti-myogenic agent. In accordance with one embodiment a pharmaceutical formulation is prepared comprising a compound selected from the group consisting of baclofen, dantrolene, tizanidine. carisoprodol, chlorzoxazone, cyclobenzaprine, metaxalone, methocarbamol, and orphenadrine. In one embodiment the pharmaceutical composition is formulated for administration by any acceptable route including as an oral, injectable or topical formulation. In one embodiment the composition is formulated for oral administration. In one embodiment the composition is formulated as a topical cream or ointment that is placed in contact with the external surface of the scar. In one embodiment the topical cream or ointment can be directly applied to the scar tissue, or the scar can be covered in gauze, bandage or other matrix that releases the formulation to the patient's affected skin a time released manner.

In one embodiment a method of reducing the severity of scar tissue formation during the process of healing after a traumatic injury to the face of mammalian subject is provided. In one embodiment the method comprises the step of administering a composition comprising an anti-myogenic agent to a subject in need of such treatment in conjunction with the administration of a muscle relaxant. In one embodiment the method comprises treating a human recovering from facial burns to reduce or prevent the amount of scar tissue formation during the healing process, wherein a pharmaceutical composition comprising an anti-myogenic agent and a pharmaceutically acceptable carrier is administered to the patient, followed by the administration of a pharmaceutical composition comprising a skeletal muscle relaxant and a pharmaceutically acceptable carrier. Reducing the amount of scar tissue formation includes any one of reducing the amount of contracture in the formed scar tissue, reducing the amount of collagen in the formed scar tissue, reducing the size of the scar. In one embodiment the anti-myogenic agent is Nilotinib. In one embodiment the pharmaceutical composition comprises a anti-myogenic agent as well as a skeletal muscle relaxant, including a muscle relaxant selected from the group consisting of baclofen, dantrolene, tizanidine. carisoprodol, chlorzoxazone, cyclobenzaprine, metaxalone, methocarbamol, and orphenadrine. In one embodiment the pharmaceutical compositions of the present invention are administered 2, 3, 4, 5, 6 weeks after the initial trauma to the face.

In one embodiment a pharmaceutical composition for reducing the severity of scar tissue formation during the process of healing after a traumatic injury to the face is formulated for administration by any acceptable route including as an oral, injectable or topical formulation. In one embodiment the composition is formulated for injection into the tissues on the periphery of the burn wound. In one embodiment the composition is formulated as a topical cream or ointment that is placed in contact with the external surface of the scar. In one embodiment the topical cream or ointment can be directly applied to the scar tissue or the scar can be covered in gauze, bandage or other matrix that releases the formulation to the patient's affected skin in a time released manner. In one embodiment a composition comprising an anti-myogenic agent and a skeletal muscle relaxant is formulated as a cream, ointment or time release matrix to be directly applied to facial contracture tissue formed, or in the process of being formed, after thermal trauma to facial skin.

In accordance with the present disclosure compositions comprising an anti-myogenic agent and/or a skeletal muscle relaxant can be used to treating patients suffering from maxillofacial thermal injuries. Optionally, the method of treating such patients comprises the sequential administration of an anti-myogenic agent followed by administration of a muscle relaxant. The methods disclosed herein reduce or prevent facial contracture formation to alleviate deficits associated with thermal injury of the face, including ectropion (epithelial-ocular junction), eversion of the lip (epithelial-oral junction) and oral incompetence (drooling). In accordance with one embodiment the compositions and methods disclosed herein are used in conjunction with known treatments for use on burn patients, including dermabrasion, chemical peels and laser resurfacing.

In one embodiment a composition is provided for treating facial burns, wherein the composition comprises a anti-myogenic agent, a skeletal muscle relaxant and a pharmaceutically acceptable carrier. In one embodiment the composition is formulated for topical application, including for example as a gel, ointment, lotion or cream. The topical formulation may include water, oil, alcohol or propylene glycol mixed with preservatives, emulsifiers, or absorption promoters. In one embodiment the composition is prepared as a gel or other matrix that releases the active agent in a time release manner. In one embodiment the composition is prepared as a transdermal patch or bandage that is applied to the scar.

In accordance with embodiment 1, a method of treating facial skin injury in mammalian subject is provided wherein the method comprises the step of administering a composition comprising an anti-myogenic agent in conjunction with the administration of skeletal muscle relaxant to the afflicted tissues of a subject in need of such treatment, optionally wherein the anti-myogenic agent is administered simultaneously with the skeletal muscle relaxant, or where the anti-myogenic agent is administered first and the skeletal muscle relaxant is administered second after a predetermined length of time has passed.

In accordance with embodiment 2 the method of embodiment 1 is provided wherein the mammalian subject is a human.

In accordance with embodiment 3 the method of embodiment 1 or 2 is provided wherein the treatment is administered during the healing phase after injury to facial tissues, optionally wherein the injury is a maxillofacial thermal injury.

In accordance with embodiment 4 the method of embodiment of any one of embodiments 1-3 is provided wherein the treatment is administered to reduce contractures associated with existing facial scars resulting from thermal injury to the face.

In accordance with embodiment 5 the method of any one of embodiments 1-4 is provided wherein the anti-myogenic agent and the skeletal muscle relaxant are administered simultaneously.

In accordance with embodiment 6 the method of any one of embodiments 1-4 is provided wherein the anti-myogenic agent is administered first followed by the administration of the skeletal muscle relaxant.

In accordance with embodiment 7 the method of any one of embodiments 1-6 is provided wherein the administration of the anti-myogenic agent is discontinued prior to administration of the skeletal muscle relaxant.

In accordance with embodiment 8 the method of any one of embodiments 1-7 is provided wherein the administration of the anti-myogenic agent is continued during the subsequent administration of the skeletal muscle relaxant.

In accordance with embodiment 9 the method of any one of embodiments 1-8 is provided wherein the anti-myogenic agent and skeletal muscle relaxant are administered by injection into said afflicted tissues, optionally via intradermal injection.

In accordance with embodiment 10 the method of any one of embodiments 1-9 is provided wherein a wound dressing comprising hydrolyzed collagen is applied to the surface of said afflicted tissues after administration of the anti-myogenic agent or skeletal muscle relaxant.

In accordance with embodiment 11 the method of any one of embodiments 1-10 is provided wherein the muscle relaxant is a compound selected from the group consisting of baclofen, dantrolene, tizanidine, carisoprodol, chlorzoxazone, cyclobenzaprine, metaxalone, methocarbamol, and orphenadrine and the anti-mycogenic agent is selected from the group consisting of Nilotinib, stromal interaction molecule (STIM) inhibitors and estrogen-related receptor alpha inhibitors.

In accordance with embodiment 12 the method of any one of embodiments 1-11 is provided wherein said facial injuries are the result of a thermal burn.

In accordance with embodiment 13 the method of any one of embodiments 1-12 is provided wherein the anti-myogenic agent and skeletal muscle relaxant are injected into skin along edges of a healing wound or proximal to a facial skin contracture.

In accordance with embodiment 14 the method of any one of embodiments 1-12 is provided wherein the anti-myogenic agent and skeletal muscle relaxant are formulated as topical creams or ointments that are applied directly to the external surface of the contracture.

In accordance with embodiment 15 the method of any one of embodiments 1-14 is provided wherein the method comprises the administration of Nilotinib as the anti-myogenic agent.

In accordance with embodiment 16 the method of any one of embodiments 1-15 is provided wherein the method comprises the administration of baclofen as the muscle relaxant.

In accordance with embodiment 17 a method of reducing the severity of contracture on the face of a mammalian subject during the healing phase after injury to the face is provided wherein the method comprises the step of administering a first composition comprising an anti-myogenic agent (but lacking a muscle relaxant) to a subject in need of such treatment followed by the administration of a second composition comprising a muscle relaxant.

In accordance with embodiment 18 the method of any one of embodiments 1-17 is provided wherein the mammalian subject is a human.

In accordance with embodiment 19 the method of any one of embodiments 1-18 is provided further comprising the step of applying a wound dressing to the surface of the contracture, wherein the wound dressing comprises hydrolyzed collagen.

In accordance with embodiment 20 the method of any one of embodiments 17-19 is provided wherein said anti-myogenic agent is Nilotinib.

In accordance with embodiment 21 the method of any one of embodiments 17-20 is provided wherein the muscle relaxant is a compound selected from the group consisting of baclofen, dantrolene, tizanidine. carisoprodol, chlorzoxazone, cyclobenzaprine, metaxalone, methocarbamol, and orphenadrine.

In accordance with embodiment 22 the method of any one of embodiments 1-21 is provided wherein said facial injury is a facial thermal burn.

In accordance with embodiment 23 the method of any one of embodiments 1-22 is provided wherein the facial thermal burn is a third degree burn or more severe burn.

In accordance with embodiment 24 the method of any one of embodiments 17-23 is provided wherein said composition is formulated as a topical cream or ointment that is applied directly to the external surface of the healing tissues of the damaged facial tissue.

### EXAMPLE 1

### Wound Healing in Porcine Maxillo-facial Thermal Trauma Model

### Materials and Methods

White pigs were subjected to severe burn trauma in the facial area (˜50% of the face) or on dorsum 6 (2″×2″) burn wounds. Progression of burn wound healing were followed till day 84 using non-invasive imaging: a) laser speckle microperfusion imaging (LSI); b) harmonic ultrasound imaging with Doppler (HUSD) for tissue stiffness and blood supply; and c) computed tomography (CT) for 3D reconstruction of the facial soft tissues and bone. Additionally, wound inflammation, angiogenesis and remodeling was examined using standard immunohistochemistry. Laser capture microdissection (LCM) of the epithelium on day 84 post burn was performed followed by whole genome RNA-sequencing.

CT and ultrasound imaging established sixth degree burn with bone involvement showing severe deficits including ectropion, oral eversion and contracture, excessive scarring as well as drooling during eating. All of these characteristics are consistent with manifestations commonly noted in humans with facial burn. Intense facial contracture was evident at day 84 post-burn. Vascular and bone deficits (n=7) were visualized using LSI, HUSD and CT imaging.

The specific areas of the face involving mucocutaneous junctions as opposed to scar healed in a different manner. RNA seq (n=6 each group; p<0.001) and bioinformatics analysis of the LCM captured epithelium revealed differential expression of a unique sets of genes following facial burn trauma that was not present in the contracture-related genes expressed following back injury in healing epithelium. Highly significantly changes were noted in the pathways associated with contraction, morphogenesis, cytoskeleton that were uniquely upregulated in facial epithelium as compared to back.

Immunohistochemistry analysis on face scar tissues relative to back skin scar tissues revealed two proteins associated with contractile tissues (paxillin 7 (Pax7) & desmin) were highly expressed in healing dermis of the face relative to healing dermis from the back of pigs. Paxillin is a cytoskeletal protein involved in actin-membrane attachment at sites of cell adhesion to the extracellular matrix (focal adhesion) and desmin is a myofibrillar protein that is the chief intermediate filament of skeletal and cardiac muscle. Elevated expression levels of smooth muscle actin was also detected in healing epithelium of the face relative to healing epithelium from the back of pigs.

These data reveal features characteristic of human facial burns involving the mucocutaneous junctions. Histopathological analysis further revealed differential inflammatory, vascularization and scarring responses at anatomic locations. This work highlights the unique molecular signatures and pathways in facial burn contracture response that will help design adequate management strategies towards facial scar contractures.

1. A method of treating facial skin injury in a mammalian subject, said method comprising the step of administering a composition comprising an anti-myogenic agent in conjunction with the administration of skeletal muscle relaxant to the afflicted tissues of a subject in need of such treatment.

2. (canceled)

3. The method of claim 1 wherein the treatment is administered during the healing phase after injury to facial tissues, optionally wherein the injury is a maxillofacial thermal injury.

4. The method of claim 1 wherein the treatment is administered to reduce contractures associated with existing facial scars resulting from thermal injury to the face.

5. The method of claim 1 wherein the anti-myogenic agent and the skeletal muscle relaxant are administered simultaneously.

6. The method of claim 1 wherein the anti-myogenic agent is administered first followed by the administration of the skeletal muscle relaxant.

7. The method of claim 6 wherein the administration of the anti-myogenic agent is discontinued prior to administration of the skeletal muscle relaxant.

8. The method of claim 6 wherein the administration of the anti-myogenic agent is continued during the subsequent administration of the skeletal muscle relaxant.

9. The method of claim 1 wherein the anti-myogenic agent and skeletal muscle relaxant are administered by injection into said afflicted tissues, optionally via intradermal injection.

10. The method of claim 6 wherein a wound dressing comprising hydrolyzed collagen is applied to the surface of said afflicted tissues after administration of the anti-myogenic agent or skeletal muscle relaxant.

11. The method of claim 10 wherein the muscle relaxant is a compound selected from the group consisting of baclofen, dantrolene, tizanidine, carisoprodol, chlorzoxazone, cyclobenzaprine, metaxalone, methocarbamol, and orphenadrine and the anti-mycogenic agent is selected from the group consisting of Nilotinib, stromal interaction molecule (STIM) inhibitors and estrogen-related receptor alpha inhibitors.

12. The method of claim 10 wherein the mammalian subject is a human and said facial injuries are the result of a thermal burn.

13. The method of claim 10 wherein the anti-myogenic agent and skeletal muscle relaxant are injected into skin along edges of a healing wound or proximal to a facial skin contracture.

14. The method of claim 10 wherein the anti-myogenic agent and skeletal muscle relaxant are formulated as topical creams or ointments that are applied directly to the external surface of the contracture.

15. The method of claim 11 wherein the method comprises the administration of Nilotinib as the anti-myogenic agent and the administration of baclofen as the muscle relaxant.

16. (canceled)

17. A method of reducing the severity of contracture on the face of a mammalian subject during the healing phase after injury to the face, said method comprising the step of administering a first composition comprising an anti-myogenic agent but lacking a muscle relaxant to a subject in need of such treatment followed by the administration of a second composition comprising a muscle relaxant.

18. (canceled)

19. The method of claim 17 further comprising the step of applying a wound dressing to the surface of the contracture, wherein the wound dressing comprises hydrolyzed collagen.

20. The method of claim 19 wherein said anti-myogenic agent is Nilotinib.

21. The method of claim 20 wherein the muscle relaxant is a compound selected from the group consisting of baclofen, dantrolene, tizanidine. carisoprodol, chlorzoxazone, cyclobenzaprine, metaxalone, methocarbamol, and orphenadrine.

22. The method of claim 21 wherein the mammalian subject is a human, and said facial injury is a facial thermal burn, optionally a third degree burn or more severe burn.

23. (canceled)

24. The method of claim 23 wherein said composition is formulated as a topical cream or ointment that is applied directly to the external surface of the healing tissues of the damaged facial tissue.
